(19) United States
(12) Patent Application Publication
(10) Pub. No.: US 2024/0041597 A1
(43) Pub. Date: Feb. 8, 2024

(54) MEDICAL DEVICE INCLUDING ATTACHABLE COMPONENTS

(71) Applicant: BOSTON SCIENTIFIC SCIMED, INC., MAPLE GROVE, MN (US)

(72) Inventors: KEVIN ROBERT POPPE, NEW BRIGHTON, MN (US); DANIEL J. FOSTER, LINO LAKES, MN (US); CHRISTOPHER JAY SCHEFF, ELK RIVER, MN (US); BRADLEY S. SWEHLA, EAGAN, MN (US); ARTHUR CONAN DEWITT, EAGAN, MN (US)

(73) Assignee: BOSTON SCIENTIFIC SCIMED, INC., MAPLE GROVE, MN (US)

(21) Appl. No.: 18/461,284

(22) Filed: Sep. 5, 2023

Related U.S. Application Data
(63) Continuation of application No. 16/995,178, filed on Aug. 17, 2020.
(60) Provisional application No. 62/887,076, filed on Aug. 15, 2019.
(60) Provisional application No. 62/887,479, filed on Aug. 15, 2019.

(51) Int. Cl. A61F 2/24; A61F 2/95

(52) U.S. Cl. CPC A61F 2/2427; A61F 2/9517

(57) ABSTRACT

Medical devices and methods for making and using medical devices are disclosed. An example system includes an inner shaft having proximal and distal end regions and a first coupling member disposed along the distal end region, wherein the first coupling member includes a first projection and a first recess. The system also includes a support shaft having proximal and distal end regions and a second coupling member disposed along the proximal end region, wherein the second coupling member includes a second projection and a second recess. The system also includes a locking collar coupled to the inner shaft. Additionally, coupling the inner shaft to the support shaft includes placing at least a portion of the first projection into the second recess, placing at least a portion of the second projection into the first recess and positioning the locking collar along a portion of both the first and second coupling members.

### CROSS-REFERENCE TO RELATED APPLICATIONS

This application is a continuation of U.S. application Ser. No. 16/995,178, filed Aug. 17, 2020, now abandoned, which claims the benefit of priority of U.S. Provisional Application No. 62/887,479 filed Aug. 15, 2019, and U.S. Provisional Application No. 62/887,076 filed Aug. 15, 2019. The entire disclosures of which is hereby incorporated by reference.

### TECHNICAL FIELD

The present disclosure pertains to medical devices, and methods for manufacturing medical devices. More particularly, the present disclosure pertains to medical devices including an attachable inner member and attachable outer member.

### BACKGROUND

A wide variety of intracorporeal medical devices have been developed for medical use, for example, intravascular use. Some of these devices include heart valves, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example system for delivering an implantable heart valve includes
an inner shaft having a proximal end region, a distal end region and a first coupling member disposed along a portion of the distal end region, wherein the first coupling member includes a first projection and a first recess. The system also includes a support shaft having a proximal end region, a distal end region and a second coupling member disposed along a portion of the proximal end region, wherein the second coupling member includes a second projection and a second recess. The system also includes a locking collar coupled to the inner shaft. Additionally, coupling the inner shaft to the support shaft includes placing at least a portion of the first projection into the second recess, placing at least a portion of the second projection into the first recess and positioning the locking collar along a portion of both the first coupling member and the second coupling member.

Alternatively or additionally to any of the embodiments above, wherein the first projection includes a first shape configured to mate with the second recess, and wherein the second projection includes a second shape designed to mate with the first recess.

Alternatively or additionally to any of the embodiments above, wherein the first projection is designed to interlock with the second projection.

Alternatively or additionally to any of the embodiments above, wherein the locking collar is designed to translate along the inner shaft.

Alternatively or additionally to any of the embodiments above, further comprising a locking channel disposed along the distal end region of the inner shaft.

Alternatively or additionally to any of the embodiments above, wherein the locking channel extends circumferentially around the distal end region of the inner shaft.

Alternatively or additionally to any of the embodiments above, wherein the locking collar includes at least one locking tab, the locking tab designed to engage within the locking channel.

Alternatively or additionally to any of the embodiments above, wherein the locking tab is designed to engage with the locking channel while the locking collar is positioned adjacent to the first projection and the second projection.

Alternatively or additionally to any of the embodiments above, wherein the second coupling member includes a first body portion attached to a second body portion, and wherein a portion of the distal end region of the support shaft is positioned between the first body portion and the second body portion.

Another system for delivering an implantable heart valve includes a tip assembly having a distal end region and a proximal end region, a guidewire shaft coupled to the distal end region of the tip assembly, an actuation shaft having a proximal end region, a distal end region and a first coupling member disposed along a portion of the distal end region, wherein the first coupling member includes a first projection and a first recess. The system also includes a support shaft having a proximal end region, a distal end region and a second coupling member disposed along a portion of the proximal end region, wherein the second coupling member includes a second projection and a second recess. The system also includes a locking collar coupled to the actuation shaft. Additionally, coupling the actuation shaft to the support shaft includes placing the first projection into the second recess, placing the second projection into the first recess and disposing the locking collar around at least a portion of both the first coupling member and the second coupling member.

Alternatively or additionally to any of the embodiments above, wherein the first projection is incompatible with the first recess and the second projection is incompatible with the second recess.

Alternatively or additionally to any of the embodiments above, wherein the first projection includes a first shape configured to mate with the second recess, and wherein the second projection includes a second shape designed to mate with the first recess.

Alternatively or additionally to any of the embodiments above, wherein the first projection is designed to interlock with the second projection.

Alternatively or additionally to any of the embodiments above, wherein the locking collar is designed to translate along the actuation shaft.

Alternatively or additionally to any of the embodiments above, further comprising a locking channel disposed along the distal end region of the actuation shaft.

Alternatively or additionally to any of the embodiments above, wherein the locking channel extends circumferentially around the distal end region of the actuation shaft.

Alternatively or additionally to any of the embodiments above, wherein the locking collar includes at least one locking tab, the locking tab designed to engage within the locking channel.

Alternatively or additionally to any of the embodiments above, wherein the locking tab is designed to engage within the locking channel while the locking collar is positioned around at least a portion of the first projection and the second projection.

Alternatively or additionally to any of the embodiments above, wherein the second coupling member includes a first body portion attached to a second body portion, and wherein a portion of the distal end region of the support shaft is positioned between the first body portion and the second body portion.

An example method for delivering an implantable heart valve includes attaching a first coupling member of an actuation shaft to a second coupling member of a support shaft of a medical device delivery system, the medical device delivery system including the implantable heart valve, wherein attaching the first coupling member of the actuation shaft to the second coupling member of the support shaft includes positioning a projection of the first coupling member into a recess of the support shaft, and positioning a projection of the second coupling member into a recess of the first coupling member. The method also includes advancing the medical device delivery system to a target site adjacent the heart and deploying the implantable heart valve at the target site.

Alternatively or additionally to any of the embodiments above, wherein attaching the first coupling member of the actuation shaft to the second coupling member of the support shaft further includes disposing a locking collar around at least a portion of both the first coupling member and the second coupling member.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example medical device system;

FIG. 2 is a side view of the tip assembly and valve assembly spaced away from the inner shaft and exoskeleton of the medical device of FIG. 1;

FIG. 3 is a perspective view of two components of the medical device of FIG. 1;

FIG. 4 is a side view of an example connection between the two components shown in FIG. 3;

FIG. 5 is a perspective view of an example connection between the two components shown in FIG. 3;

FIG. 6 is a cross-sectional view of an example connection between the two components shown in FIG. 3;

FIG. 7 is a cross-sectional view of an example connection between the two components shown in FIG. 3;

FIG. 8 is a perspective view of two components of the medical device of FIG. 1;

FIG. 9 is a perspective view of an example connection between two components shown in FIG. 8;

FIG. 10 is a perspective view of an example connection between two components shown in FIG. 8;

FIG. 11 is a cross-sectional view of an example connection between two components shown in FIG. 8;

FIG. 12 is a schematic view of a portion of the example medical device delivery system of FIG. 1;

FIG. 13 is a schematic view of a portion of the example medical device delivery system of FIG. 1;

FIG. 14 is a schematic view of a portion of the example medical device delivery system of FIG. 1;

FIG. 15 is a perspective view of a portion of the example medical device delivery system of FIG. 1;

FIG. 16 is an enlarged view of a portion of FIG. 15., with several components removed to show internal features;

FIG. 17 is a perspective view of part of the exoskeleton forming a portion of the example medical device delivery system of FIG. 1;

FIG. 18 is a side view of part of a single link of the exoskeleton of FIG. 17, showing an example magnetic sensor;

FIG. 19 is a side view of the part of the single link of FIG. 18 with the example magnetic sensor removed;

FIG. 20 is a perspective view of the example magnetic sensor of FIG. 18;

FIG. 21 is a perspective view of another example magnetic sensor;

FIG. 22 is a perspective view of another example magnetic sensor;

FIG. 23 is a perspective view of another example single link of the exoskeleton shown in FIG. 17, including a magnetic sensor;

FIG. 24 is an exploded view of the example single link shown in FIG. 23;

FIG. 25 is an exploded view of the example magnetic sensor shown in FIG. 24;

FIG. 26 is perspective view of the magnetic sensor shown in FIG. 25;

FIG. 27 is perspective view of another example sensor assembly;

FIG. 28 is an exploded view of the sensor assembly shown in FIG. 27.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Diseases and/or medical conditions that impact the cardiovascular system are prevalent throughout the world. Traditionally, treatment of the cardiovascular system was often conducted by directly accessing the impacted part of the body. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed. For example, therapies have been developed which allow a blocked coronary artery to be accessed and treated via a percutaneous catheter (e.g., angioplasty). Such therapies have gained wide acceptance among patients and clinicians.

Some relatively common medical conditions may include or be the result of inefficiency, ineffectiveness, or complete failure of one or more of the valves within the heart. For example, failure of the aortic valve or the mitral valve can have a serious effect on a human and could lead to serious health condition and/or death if not dealt with properly. Treatment of defective heart valves poses other challenges in that the treatment often requires the repair or outright replacement of the defective valve. Such therapies may be highly invasive to the patient. Disclosed herein are medical devices that may be used for delivering a medical device to a portion of the cardiovascular system in order to diagnose, treat, and/or repair the system. At least some of the medical devices disclosed herein may be used to deliver and implant a replacement heart valve (e.g., a replacement aortic valve, replacement mitral valve, etc.). In addition, the devices disclosed herein may deliver the replacement heart valve percutaneously and, thus, may be much less invasive to the patient. The devices disclosed herein may also provide a number of additional desirable features and benefits as described in more detail below.

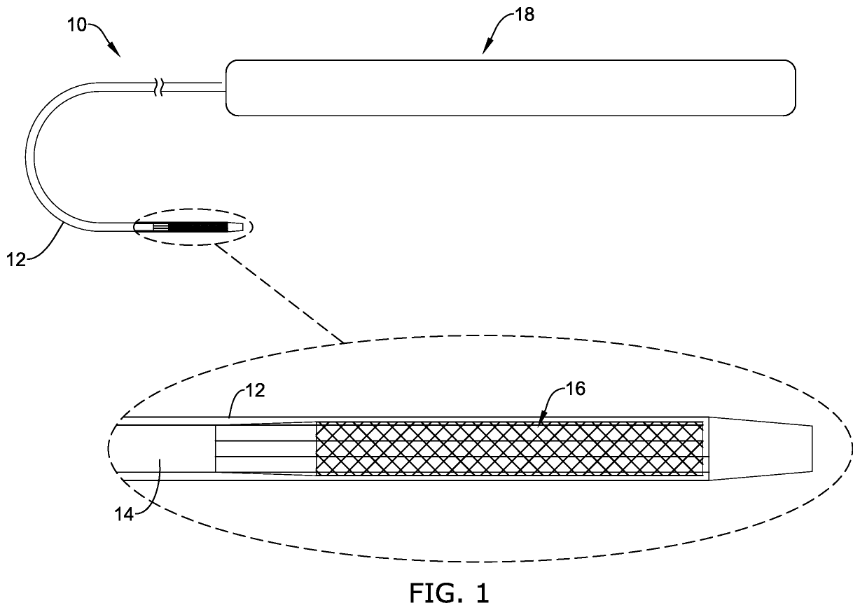

The figures illustrate selected components and/or arrangements of a medical device system 10, shown schematically in FIG. 1, for example. It should be noted that in any given figure, some features of the medical device system 10 may not be shown, or may be shown schematically, for simplicity. Additional details regarding some of the components of the medical device system 10 may be illustrated in other figures in greater detail. A medical device system 10 may be used to deliver and/or deploy a variety of medical devices to a number of locations within the anatomy. In at least some embodiments, the medical device system 10 may include a replacement heart valve delivery system (e.g., a replacement aortic valve delivery system) that can be used for percutaneous delivery of a medical implant 16 (shown in the detailed view of FIG. 1), such as a replacement/prosthetic heart valve. This, however, is not intended to be limiting as the medical device system 10 may also be used for other interventions including valve repair, valvuloplasty, delivery of an implantable medical device (e.g., such as a stent, graft, etc.), and the like, or other similar interventions.

The medical device system 10 may generally be described as a catheter system that includes an outer shaft 12, an exoskeleton 14 extending at least partially through a lumen of the outer shaft 12, and a medical implant 16 (e.g., a replacement heart valve implant) which may be coupled to the exoskeleton 14 and disposed within a lumen of the outer shaft 12 during delivery of the medical implant 16. In some embodiments, a medical device handle 18 may be disposed at a proximal end of the outer shaft 12 and/or the exoskeleton 14 and may include one or more actuation mechanisms associated therewith. In other words, one or more tubular members (e.g., the outer shaft 12, the exoskeleton 14, etc.) may extend distally from the medical device handle 18. In general, the medical device handle 18 may be designed to manipulate the position of the outer shaft 12 relative to the exoskeleton 14 and/or facilitate the deployment of the medical implant 16.

In use, the medical device system 10 may be advanced percutaneously through the vasculature to a position adjacent to an area of interest and/or a treatment location. For example, in some embodiments, the medical device system 10 may be advanced through the vasculature to a position adjacent to a defective native valve (e.g., aortic valve, mitral valve, etc.). Alternative approaches to treat a defective aortic valve and/or other heart valve(s) are also contemplated with the medical device system 10. During delivery, the medical implant 16 may be generally disposed in an elongated and low profile “delivery” configuration within the lumen and/or a distal end of the outer shaft 12, as seen schematically in FIG. 1, for example. Once positioned, the outer shaft 12 may be retracted relative to the medical implant 16 and/or the exoskeleton 14 to expose the medical implant 16. In some instances, the medical implant 16 may be self-expanding such that exposure of the medical implant 16 may deploy the medical implant 16. Alternatively, the medical implant 16 may be expanded/deployed using the medical device handle 18 in order to translate the medical implant 16 into a generally shortened and larger profile “deployed” configuration suitable for implantation within the anatomy. When the medical implant 16 is suitably deployed within the anatomy, the medical device system 10 may be disconnected, detached, and/or released from the medical implant 16 and the medical device system 10 can be removed from the vasculature, leaving the medical implant 16 in place in a “released” configuration.

It can be appreciated that during delivery and/or deployment of an implantable medical device (e.g., the medical implant 16), portions of the medical device system (e.g., the medical device system 10) may be required to be advanced through tortuous and/or narrow body lumens. Therefore, it may be desirable to utilize components and design medical delivery systems (e.g., such as the medical device system 10 and/or other medical devices) that reduce the profile of portions of the medical device while maintaining sufficient strength (compressive, torsional, etc.) and flexibility of the system as a whole.

In some instances, it may be desirable to design the medical device system 10 such that one or more device components may be disconnected from the medical device handle 18 when initially packaged (e.g., unattached to the exoskeleton 14, other inner shafts, etc.) whereby the one or more components may be subsequently coupled to the handle 18 after the packaging containing the medical device system 10 has been opened (and prior to a clinician utilizing the medical device system 10 in a medical procedure). For example, in some instances it may be desirable to package the medical implant 16 (e.g., heart valve, heart valve frame, the heart valve support structure, etc.) separately prior to performing the medical procedure. It can be appreciated that packaging the medical implant 16 (e.g., heart valve, heart valve frame, the heart valve support structure, etc.) separately may permit the medical implant 16 (including the heart valve, heart valve frame, the heart valve support structure, etc.) to be sterilized according to a different process, or kept at different temperatures, for example, than the remaining separately-packaged components of the medical device system 10.

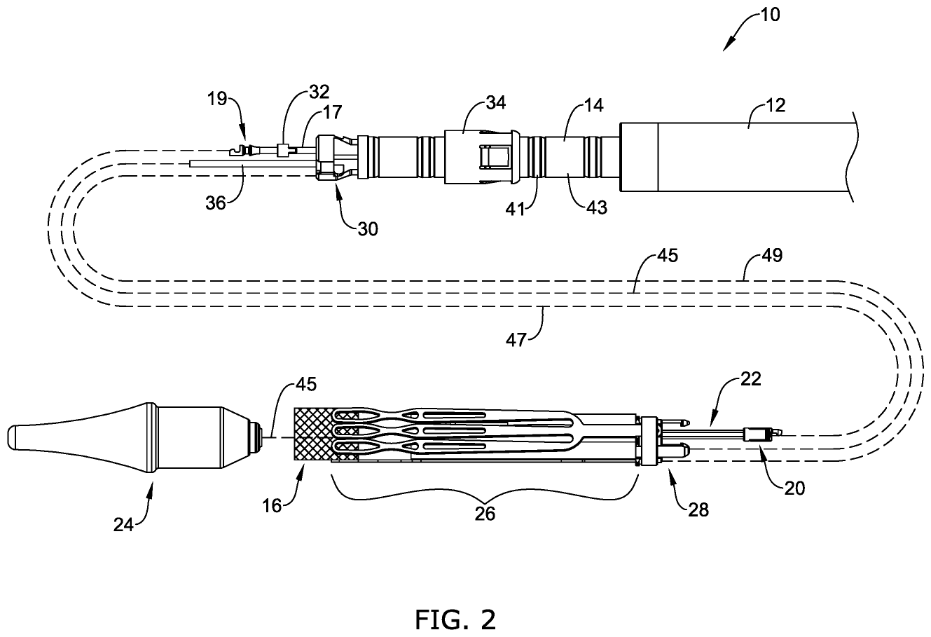

FIG. 2 shows an illustration of the medical device system 10 whereby the medical implant 16, the medical implant support structure 26 (coupled to the medical implant 16) and the tip assembly 24 are uncoupled from the handle 18 (it is noted that, for simplicity, the handle 18 is not shown in FIG. 2). It can be appreciated from FIG. 2 that any one of the medical implant 16, the medical implant support structure 26 and/or the tip assembly 24 may be packaged separately from the remaining components (e.g., handle 18, outer shaft 12, exoskeleton 14, guidewire shaft 36, etc.) of the medical device system 10, as described above.

As discussed above, FIG. 2 illustrates that the tip assembly 24 is uncoupled (e.g., unattached) from the medical implant 16, the medical implant support structure 26 and the remainder of the medical device delivery system 10. For example, in the packaging of the medical device system 10, the tip assembly may be packaged separately from the remainder of the medical device system 10. However, FIG. 2 further illustrates that the tip assembly 24 may eventually be coupled to the handle member 18 (and remainder of the medical device system 10) via a tubular guidewire member 36 (as illustrated by the dotted line 45).

In some examples, the tubular guidewire member 36 may extend proximally within the lumen of an exoskeleton 14 and couple to the handle member 18 (it is noted that the exoskeleton 14 will be discussed in greater detail below). Additionally, the tubular guidewire member 36 may include a lumen which permits a guidewire to extend and translate therein. In other words, when fully assembled, the medical device system 10 may be advanced to a target site within a body over a guidewire extending within the lumen of the tubular guidewire member 36. Further, as discussed above, the tubular guidewire member 36 may extend from the handle member 18, through the lumen of the exoskeleton 14, through the implant medical and terminate at the tip assembly 24. Additionally, to attach the tubular guidewire member 36 to the tip assembly 24, the tubular guidewire member 36 may be advanced through the medical implant support structure 26 and the medical implant 16. Further, the tip assembly 24 and the tubular guidewire member 36 may be designed such that they “quick connect” (e.g., snap, attach, engage, etc.) together. Examples of attaching the tip assembly to a tubular guidewire member 36 are disclosed in U.S. Patent Application No. XXXX (corresponding to Attorney Docket No. 2001.2057100), the entirety of which is incorporated by reference.

As discussed above, FIG. 2 further illustrates the medical implant 16 (e.g., a heart valve) coupled to a medical implant support structure 26. FIG. 2 illustrates that the medical implant 16 and the medical implant support structure 26 are uncoupled (e.g., unattached) from the remainder of the medical device delivery system 10. In the configuration shown, it can be appreciated that the medical implant support structure 26 may include one or more components and/or features which are designed to maintain the medical implant 16 in a pre-delivery configuration prior to attaching the medical implant 16 and medical implant support structure 26 to the remainder of the medical device system 10.

While FIG. 2 illustrates the medical implant 16 and the medical implant support structure 26 unattached to the remainder of the medical device system 10, it can be appreciated that the medical implant 16 and the medical implant support structure 26 may be coupled to the remainder of the medical device system 10 (e.g., handle 18) via one or more shaft members and/or coupling members (as illustrated by the dotted line 49). The coupling of the medical implant 16 and the medical implant support structure 26 to the medical device system 10 will be described below.

For example, as discussed above, FIG. 2 illustrates that the medical device system 10 may include an exoskeleton 14 extending within the outer shaft 12. The exoskeleton 14 may include one more lumens extending therein. One or more inner shafts may extend through the exoskeleton 14. For example, the exoskeleton 14 may include a lumen through which an actuation shaft 17 may extend (the actuation shaft 17 will be described in greater detail below).

Further, in some examples, the exoskeleton 14 may include a plurality of discrete members or articulating links. For example, the exoskeleton 14 may include a plurality of bead members 41 and a plurality of barrel members 43. Other discrete members are contemplated that may have differing shapes and/or configurations. In general, the discrete members (e.g., the bead members 41 and the barrel members 43) are engaged with one another and are designed to increase the compression resistance, the tension resistance, or both of the exoskeleton 14 while also affording a desirable amount of flexibility and kink resistance such that the one or more inner shafts extending through the exoskeleton can be navigated through the anatomy. The bead members 41 and the barrel members 43 may be arranged in a number of different configurations. In at least some instances, the bead members 41 and the barrel members 43 alternate along the exoskeleton 14. Other arrangements and/or patterns are contemplated. Example exoskeletons are disclosed in U.S. Patent Publication No. US20180140323, the entirety of which is incorporated by reference.

Additionally, FIG. 2 illustrates that, in some examples, the distal end of the exoskeleton 14 may include a first exoskeleton coupling member 30. As will be described in greater detail below, the first exoskeleton coupling member 30 may include one or more features which are designed to attach to a second exoskeleton coupling member 28. As further illustrated in FIG. 2, the second exoskeleton coupling member 28 may be attached to the proximal end of one or more components of the medical implant support structure 26. Therefore, it can be appreciated that coupling the first exoskeleton coupling member 30 to the second exoskeleton coupling member 28 may connect the exoskeleton 14 to the medical implant 16 via the medical implant support structure 26.

Additionally, as will be described in greater detail below, FIG. 2 illustrates that the medical device system 10 may include an exoskeleton locking collar 34. The exoskeleton locking collar 34 may be disposed along an outer surface of the exoskeleton 14. As will be described in greater detail below, the exoskeleton locking collar 34 may be utilized to couple (e.g., attach, lock, engage, etc.) the first exoskeleton coupling member 30 to the second exoskeleton coupling member 28.

It is noted that FIG. 2 illustrates the outer shaft 12 of the medical device system 10 having been retracted in a proximal direction to a position proximal of both the first exoskeleton coupling member 30, the exoskeleton locking collar 34, a portion of the actuation shaft 17 and a portion of the tubular guidewire member 36. It can be appreciated that when all the components of the medical device system 10 (including the medical implant 16, the medical implant support structure 26 and the tip assembly 24) are assembled, the outer shaft 12 may be advanced distally such that it covers the medical implant 16, the medical implant support structure 26 and a portion of the tip assembly 24.

Additionally, as discussed above, FIG. 2 illustrates that the medical device system 10 may include an actuation shaft 17 extending within a portion of the exoskeleton 14. FIG. 2 further illustrates that, in some examples, the distal end of the actuation shaft 17 may include a first actuation shaft coupling member 19. As will be described in greater detail below, the first actuation shaft coupling member 19 may include one or more features which are designed to attach to a second actuation shaft coupling member 20. As further illustrated in FIG. 2, the second actuation coupling member 20 may be attached to the proximal end of one or more translation members 22 (e.g., push-pull members). Therefore, it can be appreciated that coupling the first actuation shaft coupling member 18 to the second actuation coupling member 20 may connect the actuation shaft 17 to the medical implant 16 via the one or more translation members 22 (as illustrated by the dotted line 47).

In some examples, an operator may be able to manipulate the translation members 22 via the handle 18 (which is coupled to the translation members 22 via the actuation shaft 17, first actuation coupling member 19 and second actuation coupling member 20). For example, the handle 18 may be designed to control the translation of the translation members 22. Further, actuation of the translation members 22 may help deploy the medical implant 16 at a target site adjacent the heart. Example translation members are disclosed in U.S. patent application Ser. No. 16/396,089, the entirety of which is incorporated by reference.

Additionally, as will be described in greater detail below, FIG. 2 illustrates that the medical device system 10 may include an actuation shaft locking collar 32. The actuation shaft locking collar 32 may be disposed along an outer surface of the actuation shaft 17. As will be described in greater detail below, the actuation shaft locking collar 32 may be utilized to couple (e.g., attach, lock, engage, etc.) the first actuation shaft coupling member 18 to the second actuation coupling member 20.

In some instances, the order of connecting separately packaged components may include first advancing the guidewire shaft 36 through the medical implant. Next, the first actuation coupling member 19 may be attached to the second actuation coupling member 20. After this connection is made, the actuation shaft 17 may be retracted such that the first exoskeleton coupling member 30 may be attached to the implant support structure 26 via the second exoskeleton coupling member 28. Finally, the nosecone 24 may be attached to the distal end region of the guidewire shaft 36.

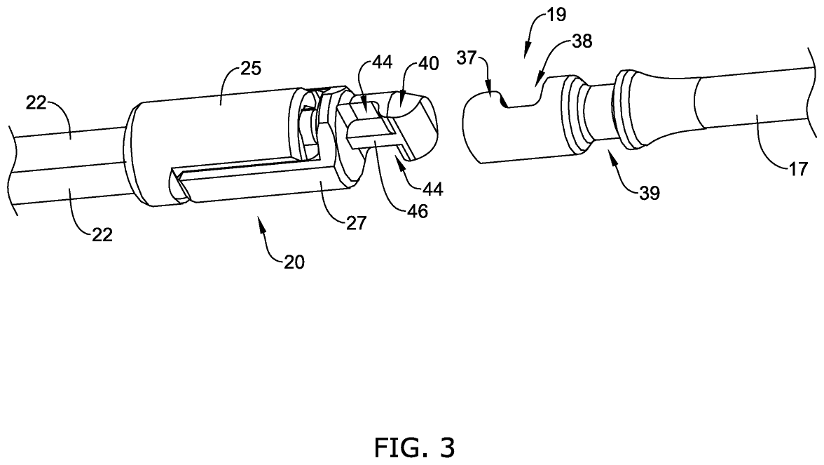

FIG. 3 is a perspective view showing the first actuation coupling member 19 and the second actuation coupling member 20. As shown in FIG. 3, the proximal end of the first actuation coupling member 19 may be attached to the distal end of the actuation shaft 17. Additionally, FIG. 3 illustrates that the first actuation coupling member 19 may include a first actuation projection 37 and a first actuation recess 38. Further, the first actuation coupling member 19 may include an actuation locking channel 39. The actuation locking channel 39 may extend around the circumference of the first actuation coupling member 19.

Additionally, FIG. 3 shows the second actuation coupling member 20 positioned adjacent to (but not yet connected to) the first actuation coupling member 19. As illustrated in FIG. 3, the second actuation coupling member 20 may include a first body portion 27 coupled to a second body portion 25. In some examples, the first body portion 27 may be attached to the second body portion via a welding process. However, this is not intended to be limiting. Rather, the first body portion 27 may be attached to the second body portion 25 using a variety of attachment techniques.

FIG. 3 further illustrates that the distal end of the second actuation coupling member 20 (including the first body portion 27 and the second body portion 25) may be attached to the proximal end of each of the translation members 22 described above. Additional details of the engagement of the first body portion 27 and the second body portion 25 with the translation members 22 is further described below.

Additionally, FIG. 3 illustrates that the proximal end of the second actuation coupling member 20 (specifically, the proximal end of the first body portion 27) may include second actuation projection 40 positioned adjacent to two second actuation recesses 44. Further, in some examples, the two second actuation recesses may be separated by a spline member 46.

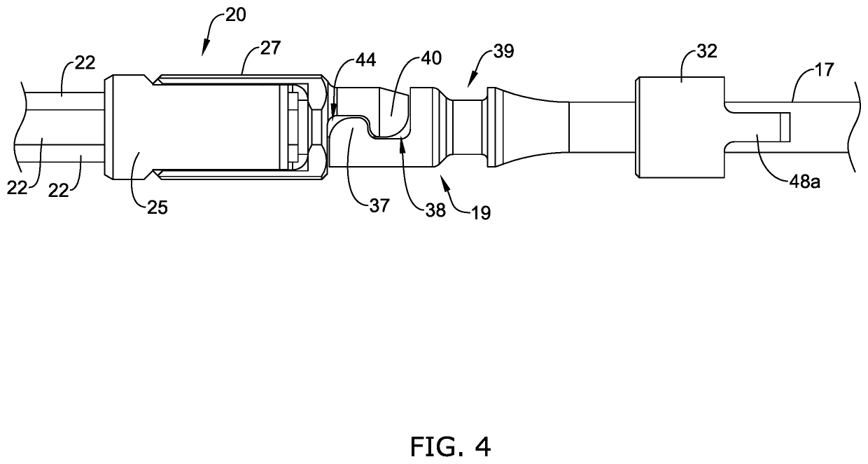

FIG. 4 illustrates a side view of the first actuation coupling member 19 coupled to the second actuation coupling member 20 (including the first body portion 27 and the second body portion 25). Specifically, FIG. 4 illustrates the first actuation projection 37 of the first actuation coupling member 19 positioned within the two second actuation recesses 44 of the second actuation coupling member 20. Additionally, FIG. 4 illustrates the second actuation projection 40 of the second actuation coupling member 20 positioned within the first actuation recess 38 of the first actuation coupling member 19. It can be appreciated that the engagement of the projections and recesses of the first actuation coupling member 19 and the second actuation coupling member 20, respectively, may resemble a “handshake” configuration of two similarly-shaped components. In other words, the projections/recesses of the first actuation coupling member 19 may be designed to mate with and engage the projections/recesses of the second actuation coupling member 20, respectively.

Additionally, FIG. 4 illustrates the actuation shaft locking collar 32 disposed along the outer surface of the actuation shaft 17. As shown in FIG. 4, the actuation shaft locking collar 32 is positioned proximal of the actuation locking channel 39. Additionally, FIG. 4 illustrates that the actuation shaft locking collar 32 may include one or more locking tabs. For example, FIG. 4 illustrates a first locking tab 48a extending proximally from the actuation shaft locking collar 32.

As described above with respect to FIG. 4, it can be appreciated that engaging the projections/recesses of the first actuation coupling member 19 with the projections/recesses of the second actuation coupling member 20, may couple the actuation shaft 17 with the translation members 22. However, it can further be appreciated that, without additional support, various forces acting on the first actuation coupling member 19 and/or the second actuation coupling member 20 may disengage the first actuation coupling member 19 from the second actuation coupling member 20. Therefore, in some instances, it may be desirable to further secure the first actuation coupling member 19 to the second actuation coupling member 20 using the actuation shaft locking collar 32.

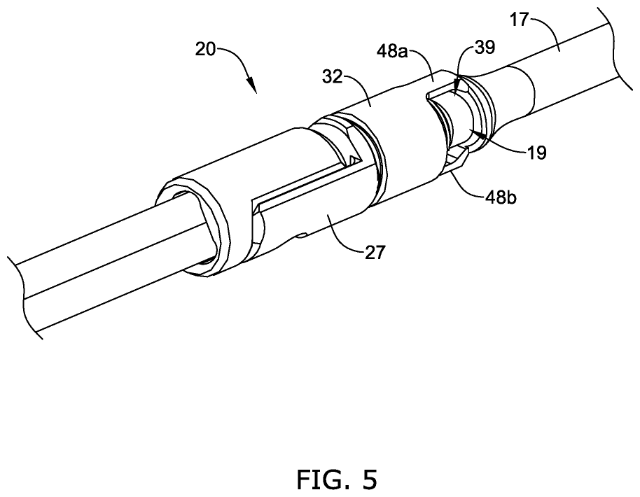

For example, FIG. 5 illustrates the actuation shaft locking collar 32 after the actuation shaft locking collar 32 has been positioned overtop the engaged projections and recesses of the first actuation coupling member 19 and the second actuation coupling member 20. Specifically, FIG. 5 illustrates the actuation shaft locking member 32 after the actuation shaft locking member 32 has been translated (e.g., slid) along the actuation shaft 17 and positioned adjacent to the first actuation coupling member 19 and the second actuation coupling member 20. Further, FIG. 5 illustrates that the actuation shaft locking collar 32 has been translated to a position in which the locking tabs 48a and 48b have been disposed within the actuation locking channel 39 of the first actuation coupling member 19.

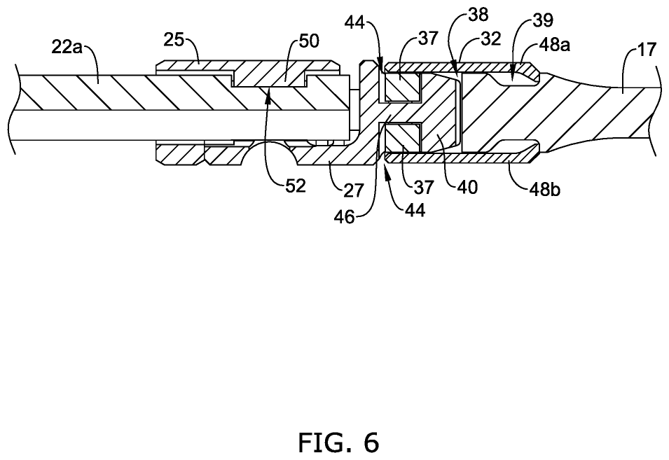

FIG. 6 illustrates a cross-sectional view of the actuation shaft locking collar 32 after the actuation shaft locking collar 32 has been positioned overtop the engaged projections and recesses of the first actuation coupling member 19 and the second actuation coupling member 20 (as illustrated and described with respect to FIG. 5 above). Specifically, FIG. 6 illustrates the first actuation projection 37 disposed within the two second actuation recesses 44 (FIG. 6 shows the first actuation projection 37 including two “teeth” which straddle the spline member 46). Further, FIG. 6 illustrates the second actuation projection 40 disposed within the first actuation recess 38.

Additionally, as described above, FIG. 6 shows the locking tabs 48a and 48b positioned within the actuation locking channel 39. It can be appreciated from FIG. 6 that the locking tabs 48a and 48b may be designed such that they bias radially inward, and therefore, they are generally designed to remain in the actuation locking channel 39 after having been disposed therein. In some instances, the translation and positioning of the actuation shaft locking collar 32 within the actuation locking channel 39 may be described as “snapping” the actuation shaft locking collar 32 (including the locking tabs 48a and 48b) within the actuation locking channel 39.

It can be further appreciated that after the actuation shaft locking member 32 has been positioned in the actuation locking channel 39, the actuation shaft 17 will remain coupled to the translation members 22 despite forces applied to the first actuation coupling member 19 and the second actuation coupling member 20. In other words, the actuation shaft locking member 32 provides a cylindrical collar that is designed to surround the projections and recesses of each of the first actuation coupling member 19 and the second actuation coupling member 20, thereby maintain their engagement as long as the locking tabs 48a and 48b remain disposed within the actuation locking channel 39.

FIG. 6 further illustrates that, in some examples, one or more projections extending radially inward from an inner surface of the second body portion 25 may engage with a recess located in the distal end of one or more of the translation members to couple the second body portion 25 with the translation member. For example, FIG. 6 illustrates a first projection 50 extending radially inward from an inner surface of the second body portion 25, whereby the projection 50 engages a first recess 52 within a translation member 22a. It can be appreciated that the engagement of the projection 50 may operate to secure the translation member 22a to the second body portion 25 (and subsequently, the actuation shaft 17 through the coupling mechanism described above with respect to the first actuation coupling member 19 and the second actuation coupling member 20).

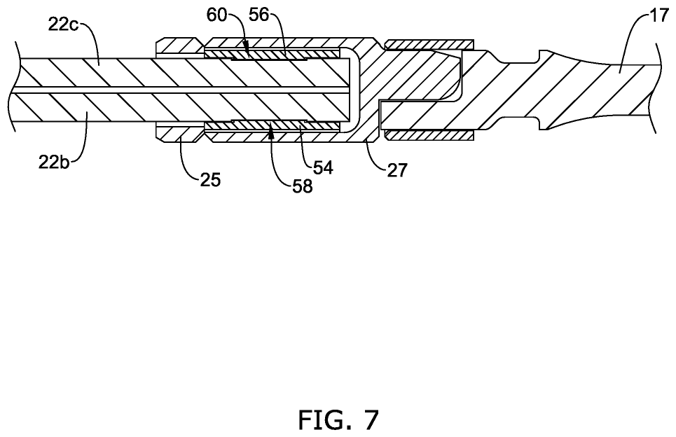

Similarly, FIG. 7 illustrates that, in some examples, a second projection 54 extending radially inward from an inner surface of the first body portion 27 may engage a second recess 58 located in the distal end of the translation member 22b to couple the first body portion 27 with the translation member 22b. Likewise, a third projection 56 extending radially inward from an inner surface of the first body portion 27 may engage a third recess 60 located in the distal end of the translation member 22c to couple the first body portion 27 with the translation member 22c. It can be appreciated that the engagement of the second projection 54 and the third projection 56 may operate to secure the translation member 22b and the translation member 22c to the first body portion 27 (and subsequently, the actuation shaft 17 through the coupling mechanism described above with respect to the first actuation coupling member 19 and the second actuation coupling member 20).

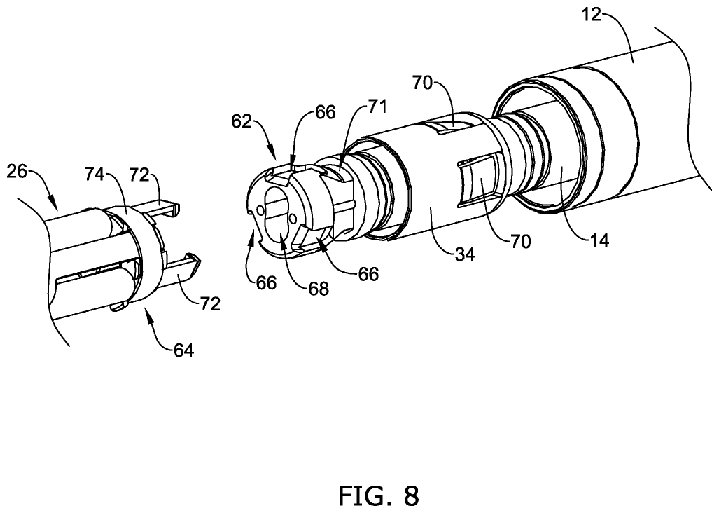

While the above discussion with respect to FIGS. 3-7 focused on the “quick-connection” mechanism of coupling the actuation shaft 17 with the translation members 22 (via the first actuation coupling member 19 and the second actuation coupling member 20), the discussion below with respect to FIGS. 8-11 will focus on the “quick-connection” mechanism of the coupling the exoskeleton 14 with the medical implant support structure 26 (which, in turn, is coupled to the medical implant 16).

FIG. 8 is a perspective view showing the first exoskeleton coupling member 62 and the second exoskeleton coupling member 64. As shown in FIG. 8, the proximal end of the first exoskeleton coupling member 62 may be attached to the distal end of the exoskeleton 14. Additionally, FIG. 8 illustrates that the first exoskeleton coupling member 62 may include a plurality of exoskeleton coupling recesses 66. The exoskeleton coupling recesses 66 may be spaced around the circumference of the first exoskeleton coupling member 62. While FIG. 8 shows three exoskeleton coupling recesses 66 spaced equidistant from one another, this is not intended to be limiting. Rather, it is contemplated that the first exoskeleton coupling member 62 may include more or less than three exoskeleton coupling recesses 66. For example, the first exoskeleton coupling member 62 may include 1, 2, 3, 4, 5, 6 or more exoskeleton coupling recesses 66, spaced equidistant or variable distances apart from one another.

Further, the first exoskeleton coupling member 62 may include an exoskeleton locking channel 71. The exoskeleton locking channel 71 may extend around the circumference of the first exoskeleton coupling member 62.

Additionally, FIG. 8 illustrates that the first exoskeleton coupling member 62 may include a lumen 68 (discussed above with respect to FIG. 2), through which one or more shafts may extend. For example, the tubular guidewire member 36 (described above, but not shown in FIG. 8), may extend through the lumen 68 of the first exoskeleton coupling member 62.

Additionally, FIG. 8 illustrates the second exoskeleton coupling member 64 positioned adjacent to (but not yet connected to) the first exoskeleton coupling member 62. As illustrated in FIG. 8, the second exoskeleton coupling member 64 may include a plurality of exoskeleton coupling fingers 72. The exoskeleton coupling fingers 72 may be spaced around the circumference of the second exoskeleton coupling member 64. While only two exoskeleton coupling fingers 72 are shown in FIG. 8, it can be appreciated that FIG. 8 is intended to depict three exoskeleton coupling fingers 72 spaced equidistant from one another (e.g., three exoskeleton coupling fingers 72 which mate with the three exoskeleton coupling recesses 66 of the first exoskeleton coupling member 62). Additionally, it is contemplated that the second exoskeleton coupling member 64 may include more or less than three exoskeleton coupling fingers 72. For example, the second exoskeleton coupling member 64 may include 1, 2, 3, 4, 5, 6 or more exoskeleton coupling fingers 72, spaced equidistant or variable distances apart from one another.

FIG. 8 further illustrates that each of the exoskeleton coupling fingers 72 may be attached to a support ring 74. The support ring 74 may be coupled to one or more components of the medical implant support structure 26.

Additionally, FIG. 8 illustrates the exoskeleton locking collar 34 disposed along the outer surface of the exoskeleton 14. As shown in FIG. 8, the exoskeleton locking collar 34 is positioned proximal of the exoskeleton locking channel 71. Additionally, FIG. 8 illustrates that the exoskeleton locking collar 34 may include one or more locking tabs 70 spaced circumferentially around the exoskeleton locking collar 34. While only two locking tabs 70 are shown in FIG. 8, this is not intended to be limiting. Rather, the exoskeleton locking collar 34 may include 1, 2, 3, 4, 5, 6 or locking tabs 70, spaced equidistant or variable distances apart from one another around the exoskeleton locking collar 34.

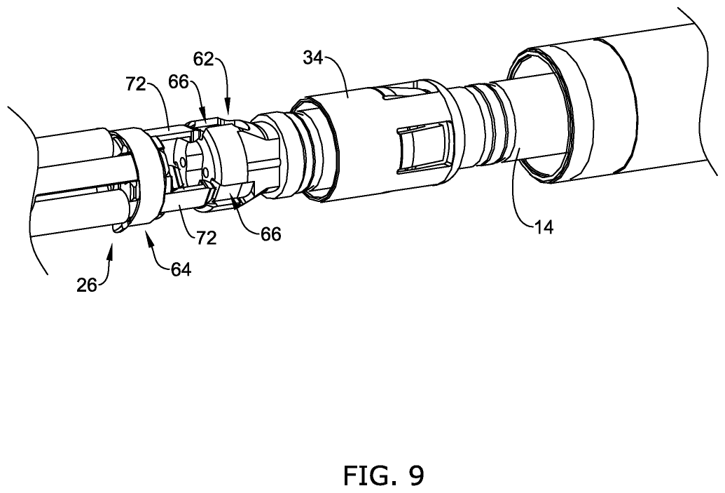

FIG. 9 illustrates a side view of the first exoskeleton coupling member 62 positioned adjacent to the coupled to the second exoskeleton coupling member 64. Specifically, FIG. 9 illustrates each of the exoskeleton coupling fingers 72 of the second exoskeleton coupling member 64 aligned with each of the exoskeleton coupling recesses 66 of the first exoskeleton coupling member 62. It can be appreciated from FIG. 9 that the shape of the each of the exoskeleton coupling fingers 72 may be designed to mate with the shape of each of the exoskeleton coupling recesses 66. In other words, it can be appreciated that the exoskeleton coupling fingers 72 shown in FIG. 9 may be further advanced into the each of the exoskeleton coupling recesses 66 shown in FIG. 9, thereby engaging each of the exoskeleton coupling fingers 72 into its respective exoskeleton coupling recesses 66.

It can be appreciated that engaging the exoskeleton coupling fingers 72 with each of the exoskeleton coupling recesses 66 may couple the exoskeleton 14 with the medical implant support structure 26. However, it can further be appreciated that, without additional support, various forces acting on the first exoskeleton coupling member 62 and/or the second exoskeleton coupling member 64 may disengage the first exoskeleton coupling member 62 from the second exoskeleton coupling member 64. Therefore, in some instances, it may be desirable to further secure the first exoskeleton coupling member 62 to the second exoskeleton coupling member 64 using the exoskeleton locking collar 34.

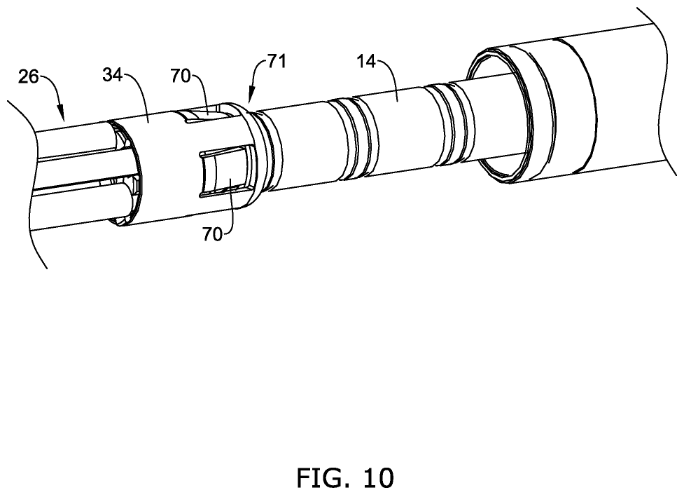

For example, FIG. 10 illustrates the exoskeleton locking collar 34 after it has been positioned overtop the exoskeleton coupling fingers 72 (which are engaged with each of the exoskeleton coupling recesses 66, as described above). Further, FIG. 10 illustrates that the exoskeleton locking collar 34 has been translated (slid) to a position in which the locking tabs 70 have been disposed within the exoskeleton locking channel 71 of the first exoskeleton coupling member 62.

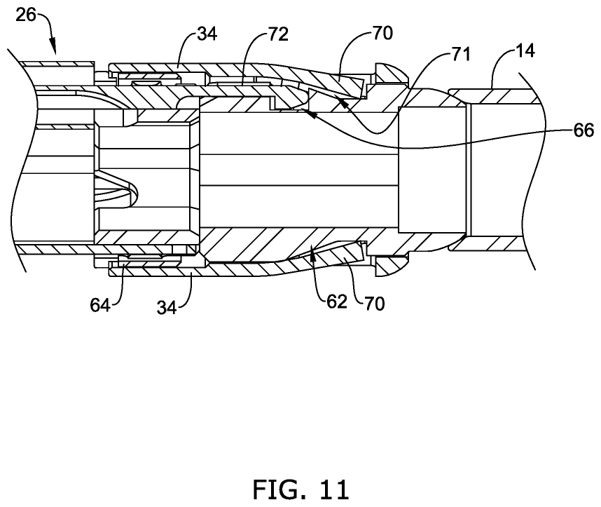

FIG. 11 illustrates a cross-sectional view of the exoskeleton locking collar 34 after it has been positioned overtop the exoskeleton coupling fingers 72 of the second exoskeleton coupling member 64 (which are engaged with the exoskeleton coupling recesses 66 of the first exoskeleton coupling member 62, as described above). Additionally, as described above, FIG. 11 shows the locking tabs 70 positioned within the exoskeleton locking channel 71. It can be appreciated from FIG. 11 that the locking tabs 70 may be designed such that they bias radially inward, and therefore, they are generally designed to remain in the exoskeleton locking channel 71 after having been positioned therein. In some instances, the translation and positioning of the exoskeleton locking collar 34 within the exoskeleton locking channel 71 may be described as “snapping” the exoskeleton locking collar 34 (including the locking tabs 70) within the exoskeleton locking channel 71.

It can be further appreciated that after the exoskeleton locking collar 34 has been positioned in the exoskeleton locking channel 71, the exoskeleton 14 will remain coupled to the medical implant support structure 26 despite various forces applied to the first exoskeleton coupling member 62 and the second exoskeleton coupling member 64. In other words, the exoskeleton locking collar 34 provides a cylindrical collar that is designed to surround the exoskeleton coupling fingers 82, thereby maintaining their engagement within the exoskeleton coupling recesses 66 as long as the locking tabs 80 remain disposed within the exoskeleton locking channel 71.

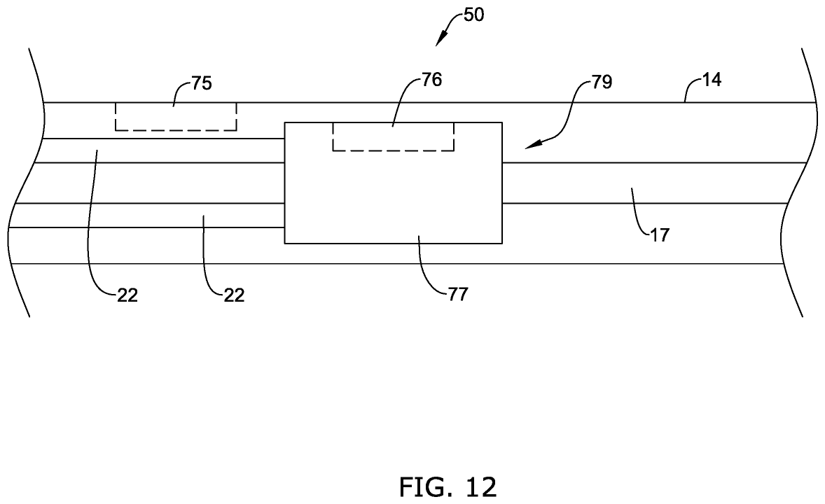

In some instances, it may be beneficial to have an indication of relative position of the force translation rod 17 and/or the push pull rods 22 relative to the exoskeleton 14, as this may provide an indication of the relative position of the medical implant 16. FIG. 12 is a highly schematic illustration showing a portion of an actuation mechanism 79 disposed within the exoskeleton 14 of the example medical device 10. A sensor 75 is shown as being disposed within or near the exoskeleton 14. In some instances, the sensor 75 may be considered as being adapted to sense the presence of a coupler 77 as the coupler 77 moves past the sensor 75 (the coupler 77 may be designed to couple the force translation rod 17 to one or more of the push pull rods 22). Accordingly, the sensor 75 may be considered as being a coupler detector, as it were. A variety of different sensors may be used as the sensor 75. In some instances, the sensor 75 may be a magnetic sensor.

Accordingly, and in some instances, the coupler 77 may include a magnetic component 76 that is secured relative to the coupler 77 such that the magnetic component 76 moves axially as the coupler 77 moves axially relative to the exoskeleton 14. In some cases, this may be reversed, with the sensor 75 secured relative to the coupler 77 while the magnetic component 76 is secured relative to the exoskeleton 14. The magnetic component 76 may be a magnet, for example. In some instances, the magnet may have a North pole and a South pole, and may be disposed relative to the coupler 77 such that the North pole and the South pole are axially aligned with the force transmission rod 17 and the push pull rods 22. As a result, the North pole and the South pole may sequentially pass the magnetic sensor 75 as the coupler 77 moves axially relative to the magnetic sensor 75. A single-pole magnet configuration will give a single peak signal as measured by the magnetic sensor 75. Alternatively, a diametrally-magnetized permanent magnet will also yield a single-peak signal.

In some cases, a double-pole assembly that includes two magnets that are mechanically connected in an anti-parallel fashion will produce a double-peak signal with negative and positive peaks being output from the magnetic sensor 75. An anti-parallel configuration means that either the two north poles of each magnet face each other, or the two south poles of each magnet face each other. The two magnets may be mechanically connected using any suitable manner, such as but not limited to an adhesive such as an epoxy, a mechanical fastener or any other mechanical connection. This configuration will enable the system to sense direction of travel of the magnetic component 76 relative to the magnetic sensor 75. For example, it may be possible to determine direction of travel by whether the negative peak or the positive peak is detected first.

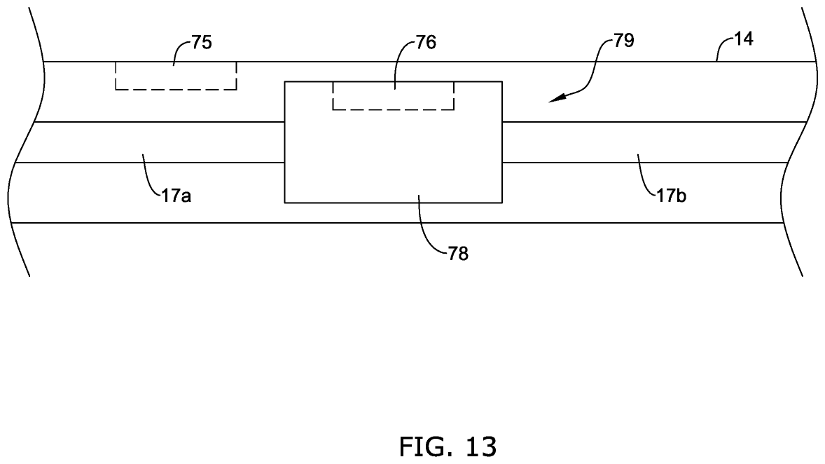

FIG. 13 is a highly schematic illustration showing the example actuation mechanism 79 disposed within the exoskeleton 14. In particular, FIG. 13 shows the force transmission rod 17 as including a distal force transmission rod 17a extending distally from a swivel connector 78 and a proximal force transmission rod 17b extending proximally from the swivel connector 78. As discussed above, the sensor 75 is shown as being disposed within or near the exoskeleton 14, at a position at which the sensor 75 is adapted to sense the presence of the swivel connector 78 and the swivel connector 78 moves past the sensor 75. A variety of different sensors may be used as the sensor 75. In some instances, the sensor 75 may be a magnetic sensor. As shown in FIG. 13, the swivel connector 78 may include the magnetic component 76.

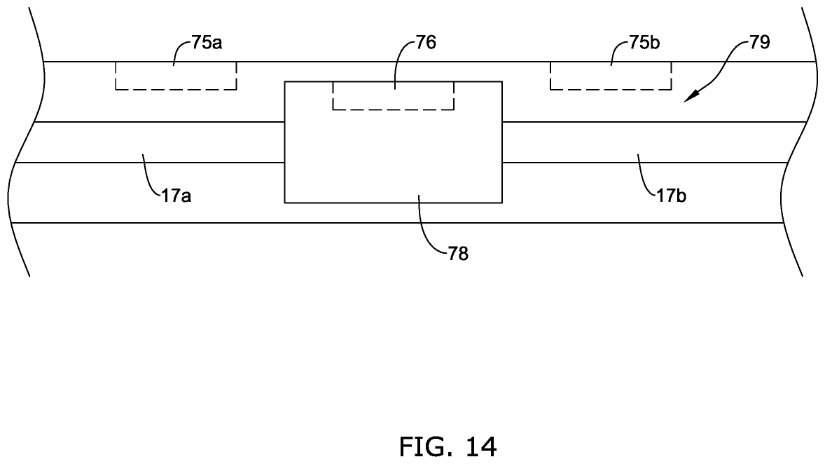

FIG. 14 is a highly schematic illustration showing the example actuation mechanism 79 disposed within the exoskeleton 14 with a pair of magnetic sensors. FIG. 14 shows a first magnetic sensor 75a and a second magnetic sensor 75b. The outputs of the magnetic sensors 75a and 75b may, for example, be connected differentially, which can be referred to as a gradiometer. This makes the sensor sensitive to magnetic fields that have non-zero spatial gradients and insensitive to spatially uniform magnetic fields. According, a gradiometer may have higher immunity to external magnetic fields resulting from the presence of magnetized objects and/or permanent magnets that may be located at a relatively large distance compared to the distance between the magnetic sensors 75a and 75b and/or the distance between the magnetic sensors 75a, 75b and the magnetic component 76. The magnetic field due to such external objects has a very low spatial gradient whereas the magnetic field of the magnetic component 76 has a significant spatial gradient.

The magnetic sensors 75, 75a, 75b may be any of a variety of different sensors that are sensitive to changing magnetic fields, as may occur as the magnetic component 76 passes by. In some instances, the magnetic sensor 75 may be a magnetoresistive sensor. Illustrative but non-limiting examples of suitable magnetoresistive sensors include anisotropic magnetoresistive (AMR) sensors, giant magnetoresistive (GMR) sensors, tunnel magnetoresistive (TMR) sensors, colossal magnetoresistive (CMR) sensors and extraordinary magnetoresistive (EMR) sensors. In some cases, a TMR sensor may be used as the magnetic sensor 75, 75a, 75b as a TMR sensor may have the best signal-to-noise ratio, particularly when used within the medical device 10. As another example, a Hall effect sensor may be used. A flux gate sensor may also be used. In some instances, the magnetic sensor 75, 75a, 75b may also be a magnetoimpedance sensor.

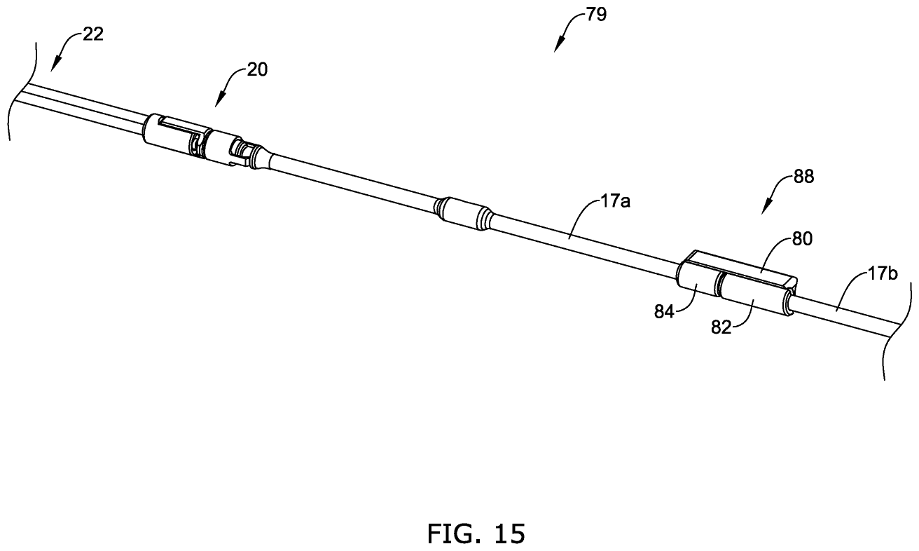

FIG. 15 is a perspective view showing additional components of the example actuation mechanism 79 described above. For example, FIG. 15 illustrates the distal force transmission rod 17a extending from the second actuation coupling member 20 to a swivel connector 88. It can be appreciated that the swivel connector 88 shown in FIG. 15 may be considered an example of the swivel connector 78 schematically shown in FIG. 7 and described above. As illustrated, the swivel connector 88 may include a saddle fitting 80, a swivel sleeve 82 and a sleeve 84.

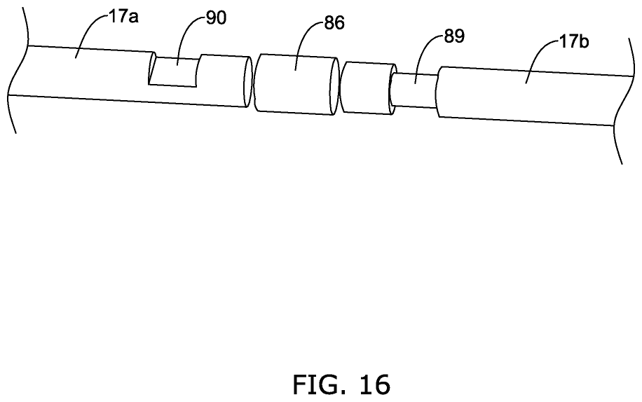

FIG. 16 shows the swivel connector 88 with the saddle fitting 80, the swivel sleeve 82 and the sleeve 84 removed in order to illustrate placement of a magnet 86 relative to the distal force transmission rod 17a and the proximal force transmission rod 17b. For example, FIG. 16 illustrates that the magnet 86 may be positioned between the distal force transmission rod 17a and the proximal force transmission rod 17b.

While FIG. 16 illustrates the magnet 86 positioned between the distal force transmission rod 17a and the proximal force transmission rod 17b, it can be appreciated that, in other examples, the sensor 75 (described above) may be positioned between the distal force transmission rod 17a and the proximal force transmission rod 17b while the magnetic component 76 may be secured to the exoskeleton 14.

It can be appreciated that the magnet 86 may be an example of the magnet 76 shown schematically in FIG. 6 and FIG. 7, described above. In some cases, as shown, the proximal force transmission rod 17b may include an annular groove 89 formed in a distal region of the proximal force transmission rod 17b as well as a recess 90 that is formed in a proximal region of the distal force transmission rod 17a. In some cases, the saddle fitting 80 includes one or more features that engage the annular groove 88 as well as the recess 90, although this is not required in all examples.

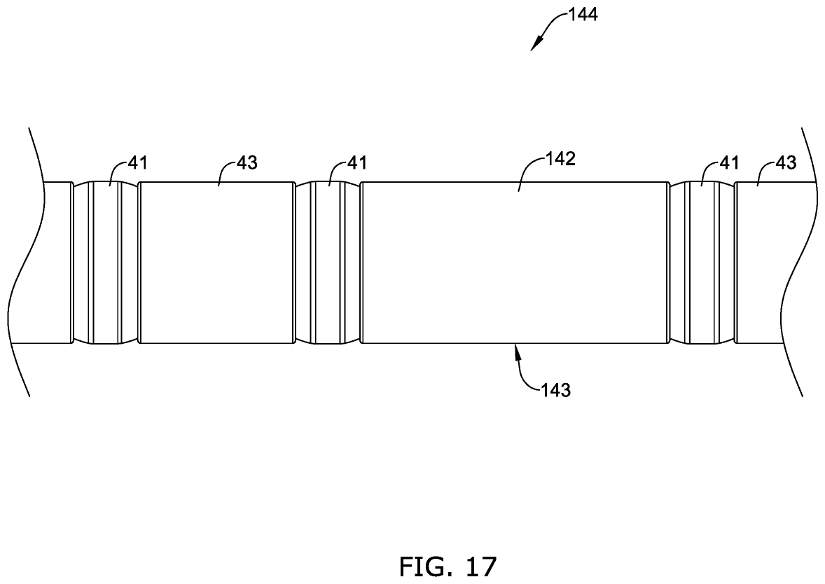

FIG. 17 is a perspective view of a portion of the exoskeleton 144. The alternating beads 41 and barrels 43 can be seen. FIG. 17 shows a link 143 that may be considered as being one of the barrels 43. In some cases, the link 143 may be longer than at least some of the others of the barrels 43, but this is not required. In some cases, the link 143 may be used to house the magnetic sensor 75. Additionally, FIG. 17 illustrates that the link 143 may include an outer housing 142. As will be described in greater detail below, the outer housing 142 may cover one or more components positioned underneath (e.g., radially inward) of the outer housing.

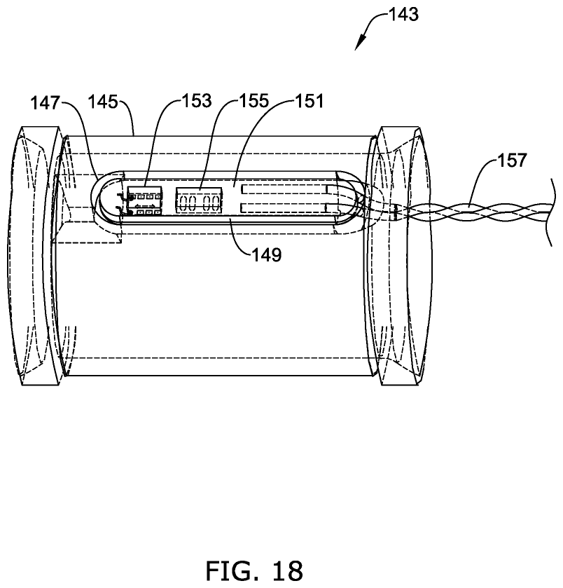

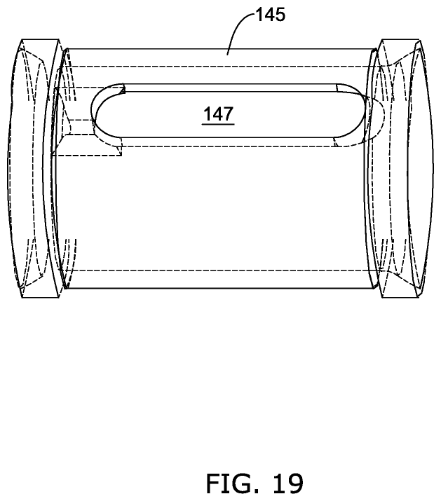

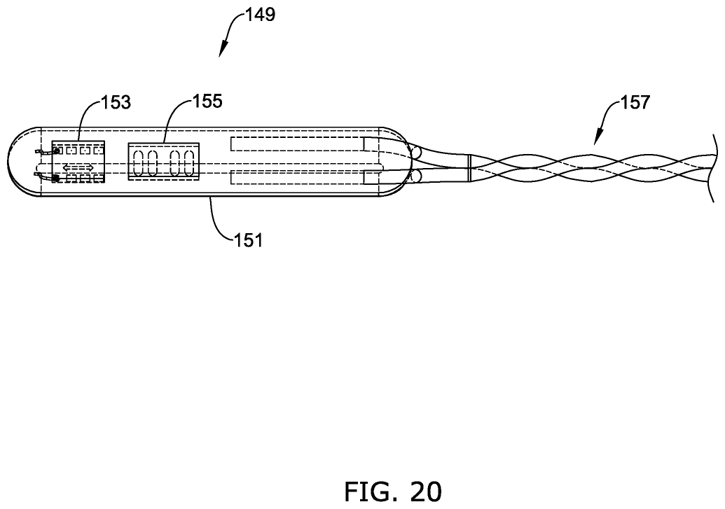

FIG. 18 is a side view of the link 143 with the housing 142 removed in order to illustrate an example magnetic sensor 149 positioned underneath the housing 142. The magnetic sensor 149 may be considered as being an example of a magnetic sensor described herein (e.g., the magnetic sensor 75 shown in FIG. 6 and FIG. 7). For clarity, FIG. 19 shows the link 143 with the magnetic sensor 149 removed while FIG. 20 is a perspective view of the magnetic sensor 149.

FIG. 18 illustrates that the link 143 may further include a socket link 145 that is disposed underneath the housing 142. It will be appreciated that the socket link 145 may form the structural portion of the link 143. As best illustrated in FIG. 19, the socket link 145 may include a void 147 that is adapted to accommodate the magnetic sensor 149 therein. In some instances, as shown, the magnetic sensor 149 may include a circuit board 151. A sensor 153 (which may for example be a TMR sensor) is operably coupled to the circuit board 151. A diode array and capacitor 155 is also operably coupled to the circuit board 151 in order to protect the sensor 153 from electrostatic discharge (ESD). ESD protection is useful since the sensor 153 can be directly attached to the circuit board 151. The capacitor aids in noise immunity by improving the stability of electrical signals going to the sensor 153. It will be appreciated that as the magnetic component 76 (FIG. 6 and FIG. 7) approaches the magnetic sensor 149, the magnetic sensor 149 may be adapted to output a signal to a remote controller (such as in the handle 15) in order to provide an indication of the relative position of the coupler 77, and hence the relative position of the medical implant 16. In some cases, an electrical conductor 157 may be operably coupled with the circuit board 151 and may extend proximally from the circuit board 151. While the electrical conductor 157 is illustrated as being a twisted pair of wires, this is not required in all cases. For example, the electrical conductor 157 may be a coaxially aligned pair of wires. The electrical conductor 157 may include three, four or more distinct electrical wires. In some cases, the electrical conductor 157 may be a flex circuit. These are just examples, and are not intended to be limiting.

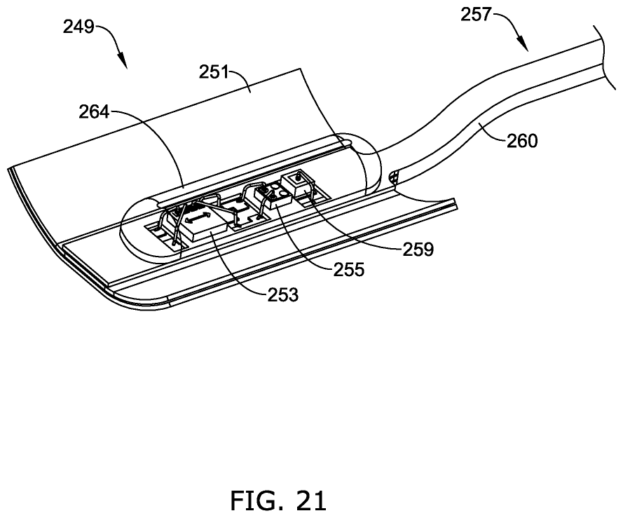

FIG. 21 is a perspective view of another example magnetic sensor 249. In some instances, as shown, the magnetic sensor 249 may include a circuit board 251. A sensor 253 (which may for example be a TMR sensor) is operably coupled to the circuit board 251 via several wirebonds. A diode 255 is also operably coupled to the circuit board 251 via several wirebonds in order to protect the sensor 253 from electrostatic discharge. In some cases, while not shown, the circuit board 251 may also include a capacitor 259 that may be coupled to the circuit board 251 via several wirebonds. In this example, the circuit board 251 includes a ribbon portion 260 that extends proximally to the handle 15 (FIG. 1). As will be appreciated, the ribbon portion 260 includes two electrical traces that function as an electrical conductor 257, much as the twisted pair of wires in FIG. 20 functions as the electrical conductor 157. In some instances, as shown, the circuit board 251 may be encapsulated within an encapsulant 264, illustrated as being transparent in order to view features on the circuit board 251. The encapsulant 264 may be a single material, or may include two or more layers. For example, the encapsulant 264 may include a first layer that is electrically insulating but not biocompatible, followed by a second layer that is biocompatible.

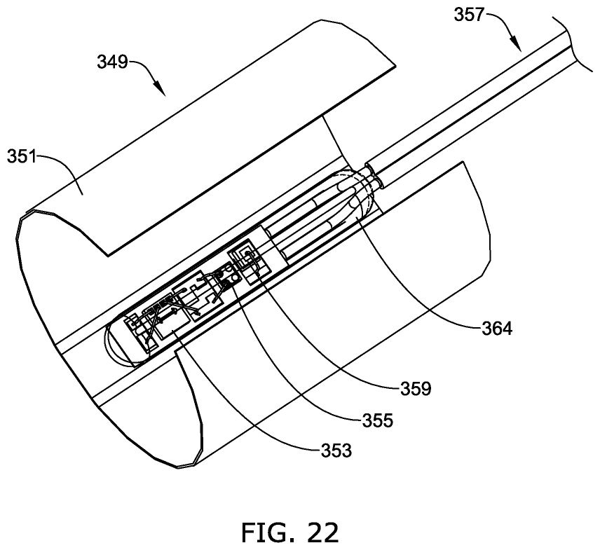

FIG. 22 is a perspective view of an example magnetic sensor 349. In some instances, as shown, the magnetic sensor 349 may include a circuit board 351. A sensor 353 (which may for example be a TMR sensor) may be operably coupled to the circuit board 351 via several wirebonds. A diode 355 may also be operably coupled to the circuit board 351 via several wirebonds in order to protect the sensor 353 from electrostatic discharge. In some cases, while not shown, the circuit board 351 may also include a capacitor 359 that may be coupled to the circuit board 351 via several wirebonds. In this example, the circuit board 351 may include an electrical conductor 357 that extends proximally. In some instances, as shown, the circuit board 351 may be encapsulated within an encapsulant 364, illustrated as being transparent in order to view features on the circuit board 351. The encapsulant 364 may be a single material, or may include two or more layers. For example, the encapsulant 364 may include a first layer that is electrically insulating but not biocompatible, followed by a second layer that is biocompatible.

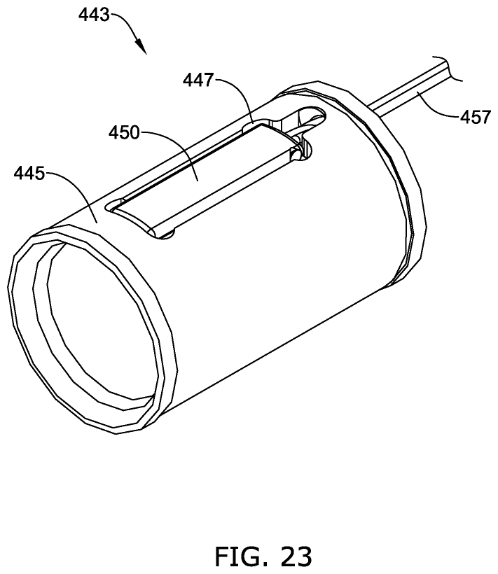

FIG. 23 illustrates another example link 443. The link 443 may be similar in form and function to other links described above. For example, the link 443 may be similar in form and function to the link 143 described above. Similar to the link 143, the link 443 may include magnetic sensor assembly 450 disposed within a void 447 of a housing 445. An electrical conductor 457 may be coupled to a proximal end region of the magnetic sensor assembly 450. Additionally, while not shown for simplicity, the link 443 may include an outer covering disposed along the housing 445. It can be appreciated that, in some examples, the covering may include a membrane, a coating, etc. designed to cover the void 447 and/or the magnetic sensor assembly 450. In other examples, the covering may be designed to include a void which aligns with the void 447. It can further be appreciated that the magnetic sensor assembly 450 may include an outer surface designed to be flush with the outer surface of the covering 445 and/or the outer surface of the housing 445 (for examples in which the link 443 does not include a covering 445).

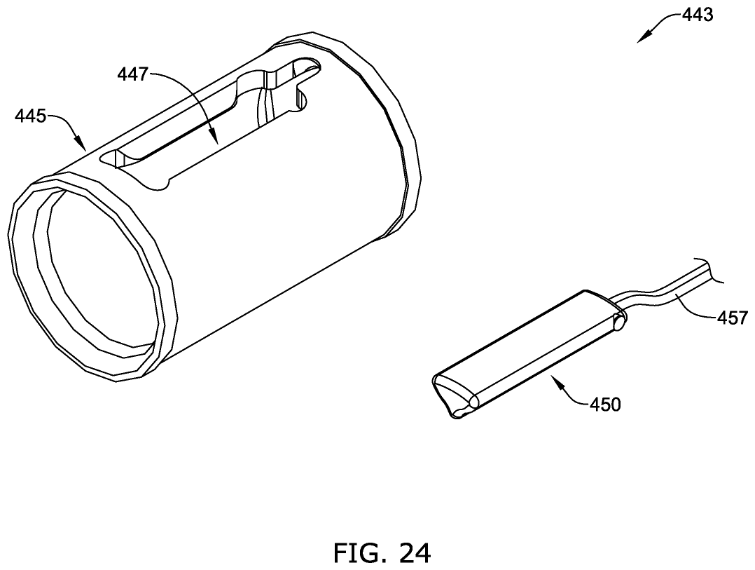

FIG. 24 is an exploded view of the link 443 described above. FIG. 24 illustrates the housing 445 including the void 447 within which the magnetic sensor assembly 450 may be disposed. FIG. 24 further illustrates the magnetic sensor assembly 450 having been removed from the void 447 in the link 443. As described above, the magnetic sensor assembly 450 may include an electrical conductor 457 extending proximally from a proximal end region of the magnetic sensor assembly 450.

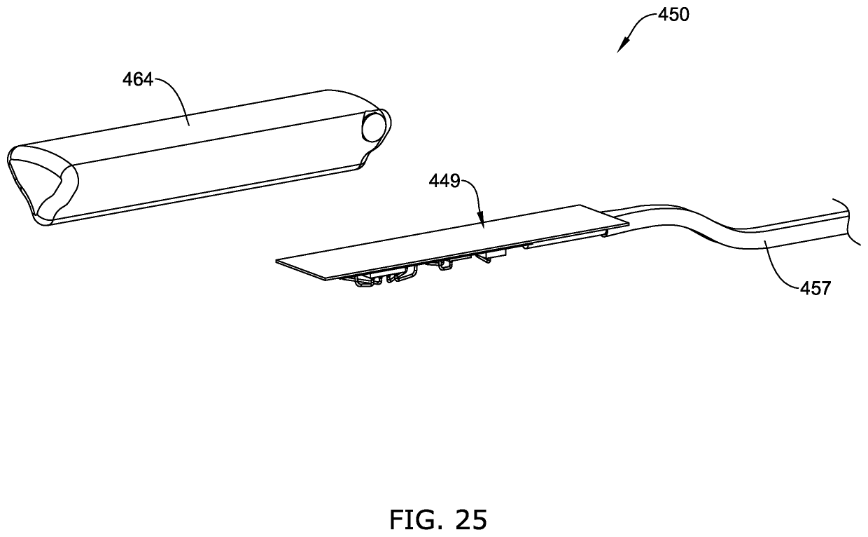

FIG. 25 illustrates an exploded view of the magnetic sensor assembly 450 described above. In particular, FIG. 25 illustrates that the magnetic sensor assembly 450 may include a magnetic sensor 449 disposed within an encapsulant 464. The encapsulant 464 may be a single material, or may include two or more layers. For example, the encapsulant 464 may include a first layer that is electrically insulating but not biocompatible, followed by a second layer that is biocompatible. Additionally, FIG. 25 illustrates that the encapsulant 464 may be shaped to fit within the void 447 in the housing 445. FIG. 25 illustrates the electrical conductor 457 extending proximally from a proximal end region of the magnetic sensor assembly 450.

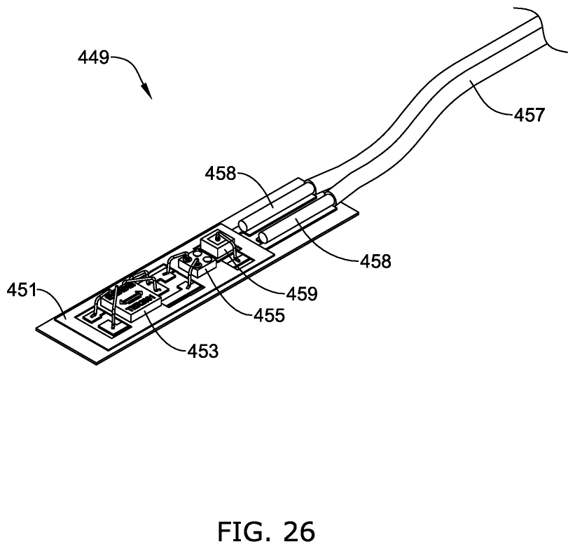

FIG. 26 illustrates the magnetic sensor 449 of the magnetic sensor assembly 450 described above. In some instances, the magnetic sensor 449 may include a circuit board 451. A sensor 453 (which may for example be a TMR sensor) may be operably coupled to the circuit board 451 via several wirebonds. A diode 455 may also be operably coupled to the circuit board 451 via several wirebonds in order to protect the sensor 453 from electrostatic discharge. In some cases, while not shown, the circuit board 451 may also include a capacitor 459 that may be coupled to the circuit board 451 via several wirebonds. In this example, the circuit board 451 may be coupled to an electrical conductor 457 that extends proximally therefrom. While the electrical conductor 457 is illustrated as being a tubular member including a pair of wires 458 extending therein, this is not required in all cases. For example, the electrical conductor 457 may be a coaxially aligned pair of wires, a twisted pair of wires or one or more electrical traces. The electrical conductor 457 may include three, four or more distinct electrical wires. In some cases, the electrical conductor 457 may be a flex circuit. These are just examples, and are not intended to be limiting.

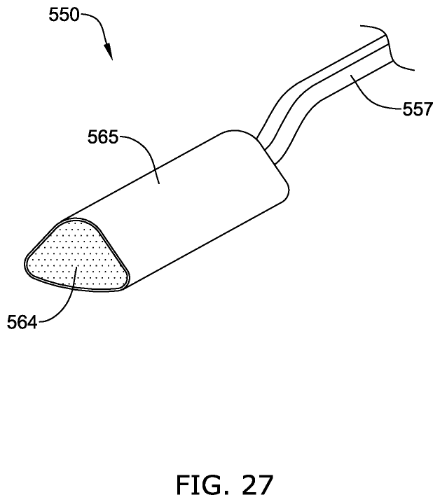

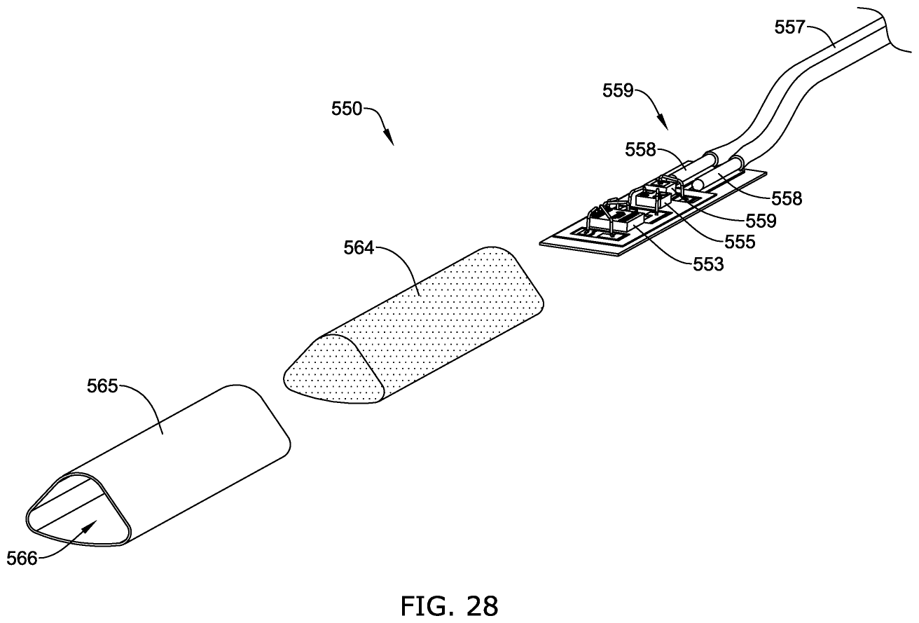

FIG. 27 illustrates another example magnetic sensor assembly 550. The sensor assembly 550 may be similar in form and function to other sensor assemblies described herein. For example, the magnetic sensor assembly 550 may be similar in form and function to the magnetic sensor assembly 450 described above. FIG. 550 illustrates the magnetic sensor assembly 550 may include an encapsulant 564 surrounded by a tubular member 565. In some examples, the tubular member may be constructed from a polymer. However, this is not intended to be limiting. For example, the tubular member 565 may be constructed from a metal, a ceramic, or other materials. Further, as will be illustrated in FIG. 28, it can be appreciated that the magnetic sensor assembly 550 may include a magnetic sensor 559 (not shown in FIG. 27, but shown in FIG. 28) which may be embedded within the encapsulant 564.

In some examples, the magnetic sensor assembly 550 may be constructed by positioning the magnetic sensor 559 (not shown in FIG. 27, but shown in FIG. 28) inside a preformed polymer tube 565 and then filling the preformed polymeric tube with the encapsulant 564. It can be appreciated that this example manufacturing methodology may embed the magnetic sensor 559 within the encapsulant 564. Additionally, the encapsulant 564 may include a first layer that is electrically insulating but not biocompatible, followed by a second layer that is biocompatible. FIG. 27 also illustrates that the encapsulant 464 may be shaped to fit within the void 447 of the example housing 445.

FIG. 28 illustrates an exploded view of the magnetic sensor assembly 50 shown in FIG. 27. Specifically, FIG. 28 illustrates the outer tubular member 565, the encapsulant 564 and the magnetic sensor 559. As discussed above, the magnetic sensor 559 may be embedded within the encapsulant 564 and both the magnetic sensor 559 and the encapsulant may be positioned within a lumen 566 of the tubular member 565.

In some instances, the magnetic sensor 549 may include a circuit board 551. A sensor 553 (which may for example be a TMR sensor) may be operably coupled to the circuit board 551 via several wirebonds. A diode 555 may also be operably coupled to the circuit board 551 via several wirebonds in order to protect the sensor 553 from electrostatic discharge. In some cases, while not shown, the circuit board 551 may also include a capacitor 559 that may be coupled to the circuit board 551 via several wirebonds. In this example, the circuit board 551 may be coupled to an electrical conductor 557 that extends proximally therefrom. While the electrical conductor 557 is illustrated as being a tubular member including a pair of wires 558 extending therein, this is not required in all cases. For example, the electrical conductor 557 may be a coaxially aligned pair of wires, a twisted pair of wires or one or more electrical traces. The electrical conductor 557 may include three, four or more distinct electrical wires. In some cases, the electrical conductor 557 may be a flex circuit. These are just examples, and are not intended to be limiting.

Some example materials that can be used for the various components of the medical device system 10 are described herein. However, this is not intended to limit the devices and methods described herein, as the other materials may be utilized for the medical device system 10 and components thereof.

Additionally, medical device system 10 and components thereof may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high density polyethylene (HDPE), polyester, Marlex high-density polyethylene, Marlex low-density polyethylene, linear low density polyethylene (for example REXELL®), ultra-high molecular weight (UHMW) polyethylene, polypropylene, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP).

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device system 10 and components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the shaft in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device system 10 and components thereof to achieve the same result.

In some embodiments, a degree of Magnetic Resonance Imaging (MM) compatibility is imparted into the shaft. For example, the medical device system 10 and components thereof may include a material that does not substantially distort the image and create substantial artifacts (e.g., gaps in the image). Certain ferromagnetic materials, for example, may not be suitable because they may create artifacts in an MRI image. The medical device system 10 and components thereof may also be made from a material that the MM machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An implantable heart valve system, comprising:
a first shaft having a proximal end region, a distal end region and a first coupling member disposed along a portion of the distal end region, wherein the first coupling member includes a first recess disposed adjacent a distal end of the first shaft and a second recess spaced apart from the first recess;
a second shaft having a proximal end region, a distal end region and a second coupling member disposed along a portion of the proximal end region, wherein the second coupling member includes a projection configured to engage the first recess on the first shaft;
a locking collar slidably coupled to the first shaft, the locking collar including at least one engagement member at a proximal end of the locking collar configured to engage the second recess;
wherein coupling the first shaft to the second shaft includes placing the projection into the first recess, and sliding the locking collar distally along a portion of the first shaft to engage the at least one engagement member with the second recess and lock the locking collar over both the first coupling member and the second coupling member; and
an implantable heart valve, wherein the second shaft includes a medical implant support structure distal of the second coupling member, the medical implant support structure configured to engage the implantable heart valve.

2. The system of claim 1, wherein the first shaft includes a plurality of articulating joints disposed proximal of the first coupling member, each articulating joint including a bead member and an adjacent a barrel member.

3. The system of claim 1, wherein the second shaft includes a medical implant support structure distal of the second coupling member, the medical implant support structure configured to engage an implantable heart valve.

4. The system of claim 1, wherein the second recess is proximal of the first recess.

5. The system of claim 1, wherein the projection includes a first shape configured to mate with the first recess, and wherein the at least one engagement member includes a second shape designed to mate with the second recess.

6. The system of claim 1, wherein the second recess is a locking channel extending circumferentially around the distal end region of the first shaft.

7. The system of claim 6, wherein the at least one engagement member includes at least one locking tab, the locking tab biased radially inward and configured to engage and remain within the locking channel once the locking collar is slid distally over the first and second coupling members.

8. The system of claim 7, wherein the locking tab includes a free proximal end biased radially inward and designed to snap into the locking channel.

9. An implantable heart valve system, comprising:
a tip assembly having a distal end region and a proximal end region;
a guidewire shaft coupled to the proximal end region of the tip assembly;
a first shaft having a proximal end region, a distal end region and a first coupling member disposed along a portion of the distal end region, wherein the first coupling member includes a first recess disposed adjacent a distal end of the first shaft and a second recess spaced apart from the first recess;
a second shaft having a proximal end region, a distal end region and a second coupling member disposed along a portion of the proximal end region, wherein the second coupling member includes a projection configured to engage the first recess; and
a locking collar coupled to the first shaft and including at least one engagement member at a proximal end of the locking collar configured to lockingly engage the second recess;
wherein coupling the first shaft to the second shaft includes placing the projection into the first recess, and disposing the locking collar over at least a portion of both the first coupling member and the second coupling member thereby locking the at least one engagement member into the second recess; and
an implantable heart valve, wherein the second shaft includes a medical implant support structure distal of the second coupling member, the medical implant support structure configured to engage the implantable heart valve.

10. The system of claim 9, wherein the first shaft includes a plurality of articulating joints disposed proximal of the first coupling member, each articulating joint including a bead member and an adjacent a barrel member.

11. The system of claim 9, wherein the second shaft includes a medical implant support structure distal of the second coupling member, the medical implant support structure configured to engage an implantable heart valve.

12. The system of claim 9, wherein the second recess is proximal of the first recess.

13. The system of claim 9, wherein the projection includes a first shape configured to mate with the first recess, and wherein the at least one engagement member includes a second shape designed to mate with the second recess.

14. The system of claim 9, wherein the locking collar is designed to translate along the first shaft.

15. The system of claim 9, wherein the second recess is a locking channel extending circumferentially around the distal end region of the first shaft.

16. The system of claim 15, wherein the at least one engagement member includes at least one locking tab, the locking tab biased radially inward and configured to engage and remain within the locking channel once the locking collar is moved over both the first coupling member and the second coupling member.

17. The system of claim 16, wherein the locking tab includes a free proximal end biased radially inward and designed to snap into the locking channel.

18. A method for delivering an implantable heart valve, the method comprising:
attaching a first coupling member of an actuation shaft to a second coupling member of a support shaft of a medical device delivery system, the medical device delivery system including the implantable heart valve;
wherein attaching the first coupling member of the actuation shaft to the second coupling member of the support shaft includes positioning a projection of the first coupling member into a recess of the support shaft, and positioning a projection of the second coupling member into a recess of the first coupling member;
advancing the medical device delivery system to a target site adjacent the heart; and
deploying the implantable heart valve at the target site.

19. The method of claim 18, wherein attaching the first coupling member of the actuation shaft to the second coupling member of the support shaft further includes disposing a locking collar around at least a portion of both the first coupling member and the second coupling member.
